(19) United States
(12) Patent Application Publication
(10) Pub. No.: US 2025/0268856 A1
(43) Pub. Date: Aug. 28, 2025

(54) Pain Relieving Method

(71) Applicant: Animal Ethics Pty Ltd, Yarra Glen (AU)

(72) Inventor: Meredith Sheil, Hunters Hill (AU)

(21) Appl. No.: 17/618,129

(22) PCT Filed: Jun. 5, 2020
(86) PCT No.: PCT/AU2020/050575

§ 371 Date: Dec. 10, 2021

(30) Foreign Application Priority Data
Jun. 11, 2019 (AU) 2019 902023

(51) Int. Cl. A61K 31/245; A61K 9/00; A61K 9/06; A61K 31/14; A61K 31/5415; A61K 47/18; A61K 47/26; A61K 47/38

(52) U.S. Cl. CPC A61K 31/245; A61K 9/0014; A61K 9/06; A61K 31/14; A61K 31/5415; A61K 47/18; A61K 47/26; A61K 47/38

(57) ABSTRACT

This invention relates to topical drug delivery formulations, formulated to carry at least one drug, be applied to an open wound, and enable systemic absorption of the at least one drug via the open wound to provide a systemic therapeutic effect for a predetermined period of time. In some embodiments, the invention concerns a topical drug delivery composition comprising a rapid onset local anaesthetic agent and/or an analgesic agent, and a drug delivery formulation, for providing both local and systemic anaesthesia and/or analgesia.

### CROSS-REFERENCE TO RELATED APPLICATIONS

This application is the United States national phase of International Application No. PCT/AU2020/050575 filed Jun. 5, 2020, and claims priority to Australian Provisional Patent Application No. 2019902023 filed Jun. 11, 2019, the disclosures of which are hereby incorporated by reference in their entirety.

### TECHNICAL FIELD

This invention relates to topical drug delivery formulations, formulated to carry at least one drug, be applied to an open wound, and enable systemic absorption of the at least one drug via the open wound to provide a systemic therapeutic effect for a predetermined period of time. In some embodiments, the invention concerns a topical drug delivery composition comprising a rapid onset local anaesthetic agent and/or an analgesic agent, and a drug delivery formulation, for providing both local and systemic anaesthesia and/or analgesia.

### INTRODUCTION

Other than for desired local effects, it is common practice to avoid application of drug products to open wounds because they may have high or unpredictable absorption, resulting in either lack of effect or systemic toxicity. Many or most drugs are directly contra-indicated for application to open wounds for this reason.

Drug products that are indicated for use on open wounds are formulated for local effect and to avoid systemic absorption. The Tri-Solfen™ product (Animal Ethics Pty Ltd), for example, is a local anaesthetic and antiseptic gel spray for use on animals to provide pain relief following mulesing, tail docking or castration. The product contains two topical local anaesthetics, being fast-acting lignocaine for immediate pain relief and long-acting bupivacaine for prolonged post-operative pain relief. The product's gel base adheres well to open wounds and acts as a barrier to environmental stimuli to improve wound healing. The Tri-Solfen™ product contains adrenalin, to both localise the anaesthetic effect and prevent systemic absorption, and thereby minimise the risk of toxicity when applied to an open wound. It was not intended for systemic delivery of a drug to achieve therapeutic levels via the open wound. See, for example, U.S. Pat. Nos. 8,960,128, 8,822,416 and 9,592,318, the entire contents of which are incorporated herein by way of cross-reference.

Morbidity from wounds however may derive from both local and systemic effects due to pain, blood loss, wound contamination, inflammation and/or sepsis. In addition to local wound treatments (such as may be provided by topical anaesthesia, haemostatic agents and/or antiseptics), there may be a requirement for systemic therapy such as for strong central or systemic analgesia, anti-inflammatory activity and/or systemic antimicrobial therapy. In this situation multimodal therapy is commonly employed, combining local treatments with systemic therapy administered via oral, IM or IV techniques.

Pain, for example, from an open wound (e.g. laceration, surgical incision, ulcer or burn) is initiated by a stimulation of traumatized nerve fibres and is intensified by a local and systemic inflammatory response that occurs over ensuing 24-48 hours resulting in local tissue swelling and oedema. Pain from an open wound is also further intensified and prolonged by a sensitization reaction of higher nerve function and initiation of the inflammatory response which also occurs over ensuing hours and days, and may lead to lower pain thresholds and prolonged hypersensitivity of surrounding tissues. Multimodal therapy is thus commonly employed to mitigate pain associated with significant wounds. Local anaesthesia (such as topical or regional local anaesthetic application or infusion), is used to block nerve conduction in combination with systemic analgesics which mitigate pain through systemic anti-inflammatory or central neurological mechanisms. Commonly used systemic analgesics include oral, intramuscular (IM) or intravenous (IV) non-steroidal anti-inflammatory drugs (NSAIDs) or opioids, which block pain through different mechanisms.

NSAIDs are commonly used systemic analgesic agents used for pain relief of wounds and other ailments. They block pain via central and local anti-inflammatory effects. The central mechanism of action augments the peripheral mechanism. This effect may be the result of interference with the formation of prostaglandins within the CNS. Alternatively, the central action may be mediated by endogenous opioid peptides or blockade of the release of serotonin (5-hydroxytryptamine; 5-HT). A mechanism involving inhibition of excitatory amino acids of N-methyl-D-aspartate receptor activation has also been proposed. At present they are used via oral or IM administration for systemic effect or separately via topical administration (on intact skin) for local effect, such as for musculoskeletal injuries such as bruises, sprains and arthritis. They are contra-indicated for use on significant open wounds, probably due to unpredictable absorption.

Opioid drugs, typified by morphine, are also commonly used for strong systemic analgesia and may be delivered via oral, IM or IV methods either alone, or in conjunction with locally acting therapy, such as for multi-modal post-surgical pain relief. Opiods produce their pharmacological actions, including analgesia, by acting on receptors located on neuronal cell membranes. The presynaptic action of opioids to inhibit neurotransmitter release is considered to be their major effect in the nervous system. They have predominantly central acting effects and are not indicated for direct application to wounds, again presumably due to concerns regarding erratic absorption.

There are situations however where such multimodal therapy is either unavailable, impractical or difficult to administer. Oral and IM administration of drug products can be problematic as delays to absorption can result in slow onset of effect as compared with IV administration. Drugs are also impacted by losses in gastro-intestinal tract. Injections and infusions on the other hand are painful, requiring skilled professionals to administer, and can be dangerous due to rapid rises in peak concentrations, as well as having shorter duration effect. A further major problem is that anaesthetic and analgesic agents need to be administered to a subject separately, usually by way of injection by a specialist, and this can be impractical, costly and inconvenient, especially when a large number of humans or animals require en-masse treatments or are located in regional or remote situations. This is particularly so in the setting of front-line military, or mass disaster situations, remote regional and rural locations and/or animal husbandry procedures.

A completely novel approach is to invent topical wound application methods and formulations to deliver systemic therapy directly via wounds (either alone, or in conjunction with local acting agents). A topical wound application combining local anaesthetics for direct rapid onset local effects with a long-acting NSAID for prolonged systemic anti-inflammatory effects for example, may result in rapid onset pain relief of prolonged duration by simultaneously targeting both local and central neural and inflammatory mechanisms of pain generation.

Wounds provide a previously unconsidered portal for systemic drug administration via topical application. Topical application of drugs to intact skin generally inhibits systemic absorption due to skin barrier effects, hence the requirement for oral, injected or other modes of systemic drug delivery. In the case of wounds however, the skin is breached, providing opportunities for systemic drug delivery. Safe and effective systemic drug delivery is consequent upon achieving systemic drug levels in the therapeutic range i.e. above levels known to be ineffective, and below levels known to be toxic. Conventional wisdom based on past evidence is that topical application of drugs to open wounds is neither safe or effective as a method to achieve therapeutic blood levels or deliver systemic therapy, primarily due to erratic absorption. The present inventor, however, has challenged this paradigm and has newly discovered that safe and effective systemic drug delivery can be achieved using topical wound application resulting in sustained systemic therapeutic drug concentrations.

Although the potential to deliver systemic acting therapy via direct topical wound applications is a novel concept and against conventional wisdom, the ability to provide both local and systemically acting agents in combination via a single topical wound formulation represents a convenient and economic method of delivering synergistic efficacies and would be of obvious welfare benefit to the target human or animal in that it could constitute a single topical application and would not require injections, additional treatments and/or multiple handling. The invention of a direct wound topical application that can provide local and systemic therapeutic effects, such as the combination of local anaesthetic agent and systemic analgesic agent is timely with the need to better manage wounds in military and disaster situations and with the global push for the provision of improved pain relief for routine husbandry procedures in animals.

### SUMMARY OF INVENTION

The inventor has made the unexpected discovery that, in some embodiments, some drug delivery formulations, applied topically to open wounds, can provide both safe and effective local and central/systemic drug delivery.

The inventor has found that, in some embodiments, systemic delivery through open wounds can be more rapid in achieving peak systemic effect than IM or oral administration.

The inventor has found that, in some embodiments, systemic delivery via open wounds can avoid the rapid peaks, risk of toxicity and the pain involved with intravascular administration as well as provide a longer release effect. This is of advantage, for example, for wound pain relief involving drugs that have both local and central/systemic effects, such as NSAIDs and anaesthetics. This is of advantage, for example, for administering antibiotics, anti-parasitic agents (eg. antifungals or antihelminthics) that have both local and central/systemic effects.

According to a first aspect of the present invention, there is provided a topical drug delivery formulation, formulated to carry at least one drug, be applied to or administered via an open wound of a subject, and enable systemic absorption of the at least one drug through the open wound to provide a therapeutic effect for a predetermined period of time.

According to a second aspect of the present invention, there is provided a topical drug delivery composition comprising:
at least one drug; and
a drug delivery formulation, formulated to carry the at least one drug, be applied to or administered via an open wound of a subject, and enable systemic absorption of the at least one drug through the open wound to provide a therapeutic effect for a predetermined period of time.

According to a third aspect of the present invention, there is provided a method of delivering at least one drug systemically to a subject via an open wound of said subject, said method comprising the step of topically applying to or administering via the open wound the drug delivery formulation of the first aspect of the invention or the drug delivery composition of the second aspect of the invention to provide a therapeutic effect for a predetermined period of time.

According to a fourth aspect of the present invention, there is provided use of at least one drug and a drug delivery formulation in the preparation of a medicament for delivering the at least one drug systemically to a subject via an open wound of said subject, wherein said drug delivery formulation is formulated to carry the at least one drug, to be topically applied to or administered via the open wound, and enable systemic absorption of the at least one drug through the open wound to provide a therapeutic effect for a predetermined period of time.

According to a fifth aspect of the present invention, there is provided a drug delivery formulation for use in carrying at least one drug, to be topically applied to or administered via an open wound of a subject, and enabling systemic absorption of the at least one drug by the subject through the open wound to provide a therapeutic effect for a predetermined period of time.

According to a sixth aspect of the present invention, there is provided a topical drug delivery composition for use in delivering at least one drug systemically to a subject via an open wound of the subject, wherein said composition comprises:
at least one drug; and
a drug delivery formulation, formulated to carry the at least one drug, to be topically applied to or administered via a significant open wound of the subject, and enable systemic absorption of the at least one drug through the significant open wound to provide a therapeutic effect for a predetermined period of time.

According to a seventh aspect of the present invention, there is provided a method of manufacturing a topical drug delivery composition, said method comprising the step of combining at least one drug with a drug delivery formulation, formulated to carry the at least one drug, to be topically applied to or administered via an open wound of the subject, and enable systemic absorption by the subject of the at least one drug through the open wound to provide a therapeutic effect for a predetermined period of time.

Any suitable type of drug or drugs can be used. There can be more than one type of drug, including two, three, four, five, six, seven, eight, nine, 10 or even more drugs.

A “drug” as defined herein is a compound, molecule, extract, mixture or other type of agent or agent combination that provides a therapeutic effect which is of benefit to the subject. Preferably the drug provides both a beneficial local and systemic effect.

The drug can be, for instance, an analgesic, anaesthetic, sedative, narcotic, anxiolytic antibiotic, anti-microbial, antifungal or anti-parasitic agent, antibody, coagulant, anticoagulant, haemostatic agent, immune globulin, vaccine, vasopressor, inotrope, alpha blocker, beta blocker, antiarrhythmic, antihistamine, antiproliferative, cytokine, cytotoxin, growth factor, interferon, steroid, hormone, lipid, demineralized bone or bone morphogenetic protein, cartilage inducing factor, oligonucleotide, polymer, polysaccharide, polypeptide, protease inhibitor, vitamin, mineral, or antiseptic agent.

Any suitable amount of drug can be used. In some embodiments, about 0.001 to 20 weight/volume % or weight/weight % or volume/volume % of agent is used (as well as all 0.001 increments between 0.001 and 20). In some embodiments, about 0.05 to 20% weight/weight or weight/volume of drug is used (as well as all 0.01 increments between 0.05 and 20). In some embodiments, about 0.1 to 10% weight/weight or weight/volume of drug is used (as well as all 0.01 increments between 0.1 and 10). The amount of drug may depend on a number of factors, including the potency of the drug, the site and nature of the open wound, the body weight of the subject et cetera.

The drug can be an anti-parasitic agent. Examples of anti-parasitic agents include: nitazoxanide, melarsoprol, eflornithine, metronidazole, tinidazole, miltefosine, mebendazole, pyrantel pamoate, thiabendazole, diethylcarbamazine, ivermectin, niclosamide, praziquantel, albendazole, praziquantel, rifampin, amphotericin B, and fumagillin.

Examples of parasitic infestations for treatment with anti-parasitic agents may be caused by the following: Ostertagia (brown stomach worm); Haemonchus (barberpole worm); Trichostrongylus (bankrupt worm); Cooperia (small intestinal worm); Nematodirus (threadneck worm); Oesophagostomum (nodular worm); Haemonchus Bunostomum (hookworm); Strongyloides (threadworm); Trichuris (whipworm); Moniezia (tapeworm); Dictyocaulus (lungworm); Helminths; Schistosomes; Flatworms (Platyhelminthes); Cestodes (tapeworms); Trematodes (flukes and blood flukes); Nematodes (roundworms); Acanthocephalins (thorny-headed worms); and, Annelids (ringed worms).

The drug can be an antibiotic. Examples of antibiotics include amoxicillin, doxycycline, cephalexin, ciprofloxacin, clindamycin, metronidazole, azithromycin, sulfamethoxazole/trimethoprim and levofloxacin.

The drug can be an antifungal. Examples of antifungals include clotrimazole, econazole, ketoconazole, miconazole, tioconazole, fluconazole, itraconazole, posaconazole, voriconazole, amphotericin B, nystatin, caspofungin, anidulafungin, micafungin, griseofulvin, terbinafine and flucytosine.

The drug can be an anaesthetic. Examples of anaesthetics potentially include tetracaine, lignocaine, chloroprocaine, mepivacaine, bupivacaine, articaine, etidocaine, levobupivacaine, prilocaine, benzocaine, ropivacaine, cocaine, oxyprocaine, hexylcaine, dibucaine, piperocaine and procaine, and pharmaceutically acceptable acids, bases and salts thereof.

The composition or drug delivery formulation preferably provides maximum analgesia with minimal risk of toxicity. The formulation of the composition may be varied, as required, for potency, speed of onset and duration of action.

In some embodiments, the drug can be a local anaesthetic agent. In some embodiments, the local anaesthetic agent can have a rapid onset of action. In some embodiments, the local anaesthetic agent can have a long duration of action. In some embodiments, the composition can comprise, or the drug delivery formulation can carry, both a local anaesthetic agent having a rapid onset of action and a local anaesthetic agent having a long duration of action. It is to be understood that some local anaesthetic agents can provide both a rapid onset of action and long duration of action, such as tetracaine/amethocaine, so the local anaesthetic agent providing a rapid onset of action and local anaesthetic agent providing a long duration of action can be one and the same.

Anaesthetic agents that usually have a rapid onset of action (usually between about 5-10 minutes) include lignocaine, prilocaine, amethocaine/tetracaine and cocaine.

Anaesthetic agents that have a much greater duration of action (usually between about 4-12 hours of anaesthesia) include bupivacaine and amethocaine/tetracaine.

Any suitable amount of anaesthetic agent can be used but preferably about 0.01-10 weight/volume % of anaesthetic agent is used (as well as all 0.01 increments between 0.01 and 10, eg. 0.01, 0.02 etc).

Any suitable amount of rapid onset anaesthetic agent can be used but preferably about 0.01-10 weight/volume % of anaesthetic agent is used (as well as all 0.01 increments between 0.01 and 10). Preferably, about 2-8 weight/volume % anaesthetic agent is used in those situations where a rapid onset of action is required (as well as all 0.01 increments between 2 and 8). More preferably, about 5% weight/volume anaesthetic agent is used.

In some embodiments, about 1-10 weight/volume % lignocaine is used (as well as all 0.01 increments between 1 and 10, eg. 0.01, 0.02 etc). In some embodiments, about 2-8 weight/volume % lignocaine is used as the anaesthetic agent in those situations where a rapid onset of action is required (as well as all 0.01 increments between 2 and 8). In some embodiments, about 5% lignocaine is used.

Any suitable amount of long duration of action anaesthetic agent can be used but preferably about 0.01-10 weight/volume % of anaesthetic agent is used (as well as all 0.01 increments between 0.01 and 10). Preferably, about 0.1-5 weight/volume % anaesthetic agent is used in those situations where a long duration of action is required (as well as all 0.01 increments between 0.1 and 5). More preferably, about 0.5% weight/volume anaesthetic agent is used.

In some embodiments, any suitable amount of bupivacaine can be used. Preferably, about 0.1-5 weight/volume % bupivacaine is used (as well as all 0.01 increments between 0.1 and 5), and more preferably about 0.5% bupivacaine.

Preferred drug examples include lignocaine hydrochloride monohydrate and bupivacaine hydrochloride monohydrate.

Other potential anaesthetic agents include: butamben, butambenpicrate, dimethisoquin hydrochloride, diperodon, diphenhydramine, dyclonine, ketamine, methapyriline, p-buthylaminobenzoic acid, 2-(die-ethylamino) ethyl ester hydrochloride, pramoxine, tripelennamine, propofol, etomidate, and barbiturates (e.g., thiopental).

The drug can be an analgesic agent or combination of agents. Potentially suitable analgesic agents include one or more of the following: acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, pranoprofen, fenoprofen, sulindac, fenbufen, clidanac, flurbiprofen, indoprofen, protizidic acid, fentiazac, tolmetin, tiaprofenic acid, bendazac, bufexemacpiroxicam, phenylbutazone, oxyphenbutazone, clofezone, pentazocine, mepirizole, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, naproxen, suprofen, piroxicam, diflunisal, meclofenamate sodium, carprofen, flunixin, tolfenamic acid and meloxicam.

In some embodiments, the analgesic agent can be a non-steroidal anti-inflammatory drug (NSAID). The NSAID can be a salicylate (e.g. aspirin (acetylsalicylic acid), diflunisal (dolobid), salicylic acid and other salicylates, salsalate (disalcid)), propionic acid derivative (e.g. ibuprofen, dexibuprofen, naproxen, fenoprofen, ketoprofen, dexketoprofen, flurbiprofen, oxaprozin, loxoprofen), acetic acid derivative (e.g. indomethacin, tolmetin, sulindac, etodolac, ketorolac, diclofenac, aceclofenac, nabumetone), enolic acid (oxicam) derivative (e.g. piroxicam, meloxicam, tenoxicam, droxicam, lornoxicam, isoxicam, phenylbutazone), anthranilic acid derivative (fenamate) (e.g. mefenamic acid, meclofenamic acid, flufenamic acid, tolfenamic acid), selective COX-2 inhibitor (e.g. celecoxib, rofecoxib, valdecoxib, parecoxib, lumiracoxib, etoricoxib, firocoxib), sulfonanilide (e.g. nimesulide), or other (e.g. clonixin, licofelone, H-harpagide in Figwort or Devil's Claw).

Any suitable amount of analgesic or anti-inflammatory agent can be used but preferably about 0.01-10 weight/volume % of agent is used (as well as all 0.01 increments between 0.01 and 10). Preferably, about 0.1% w/v of analgesic agent, such as meloxicam, is used.

The drug may be a vasoconstrictor or haemostatic agent. Examples include: amino acids; aminocaproic acid, tranexamic acid, aminomethylbenzoic acid, proteinase inhibitors; aprotinin, alfa1 antitrypsin, camostat, vitamin K and other hemostatics: vitamin K; phytomenadione, menadione, fibrinogen: human fibrinogen; local hemostatics; oxidized cellulose, tetragalacturonic acid hydroxymethylester, adrenalone, thrombin, collagen, calcium alginate, epinephrine microfibrillar collagen haemostat, chitosan hemostats, kaolin, zeolite, stypics including anhydrous aluminium sulfate; blood coagulation factors; coagulation factor IX, II, VII and X in combination (rothrombin complex concentrate), coagulation factor VIII, factor VIII inhibitor bypassing activity, coagulation factor IX, coagulation factor VII, von Willebrand factor and coagulation factor VIII in combination, coagulation factor XIII, coagulation factor VIIa, von Willebrand factor, catridecacog, coagulation factor X, susoctocog alfa, rhrombin; other systemic hemostatics; etamsylate, carbazochrome, batroxobin, romiplostim, eltrombopag, emicizumab, lusutrombopag, avatrombopag, and fostamatinib.

In the field, IV medications may not be able to be established, it can be difficult to find veins in people in hemorrhagic shock, and IM can be unpredictable or slow to have effect. An open wound may provide a rapid, simple and an effective way of delivering acute resuscitation medication. In this situation there would be delivery of a higher dose of adrenalin or resuscitation medicines. Adrenalin in acute cardiac arrest, for example, is given in 1 mg IV boluses.

The drug can be a vasopressor or inotrope. Any suitable type of vasopressor can be used. Suitable vasopressors include, for instance, adrenaline (epinephrine), noradrenalin (norepinephrine), fenylpressin, vasopressin, phenylephrine, metaraminol, a synthetic catecholamine, dobutamine, dopexamine, dopamine, levosimendan, pimobendan, nesiritide, amrinone, enoximone, milrinone, olprinone, terlipressin and ornipressin.

The drug can be an antiarrhythmic. Any suitable type of antiarrhythmic can be used. Suitable antiarrhythmics include, for instance, adenosine, ajmaline, amiodarone, adrenaline, and atropine.

The drug can be a tranquiliser or sedative. Any suitable type of tranquiliser or sedative can be used. Suitable tranquilisers and sedatives include, for instance, benzodiazepines (eg. diazepam, midazolam), butyrophenone (eg. azaperone), and phenothiazines (eg. acepromazine maleate, chlorpromazine hydrochloride, promazine hydrochloride, triflupromazine hydrochloride).

The drug can be a narcotic. Any suitable type of narcotic can be used. Suitable narcotics include, for instance, codeine, hydrocodone, oxycodone, methadone, morphine, hydromorphone, meperidine, tramadol and fentanyl.

The drug can be an anxiolytic. Any suitable type of anxiolytic can be used. Suitable anxiolytics include, for instance, benzodiazepines, eg. alprazolam, chlordiazepoxide, clonazepam, diazepam and lorazepam.

The drug can be an anticoagulant. Any suitable type of anticoagulant can be used. Suitable anticoagulants include, for instance, anticoagulant reversal agents, coumarins, indandiones, factor Xa inhibitors, heparins, thrombin inhibitors, antiplatelet agents, glycoprotein platelet inhibitors, platelet aggregation inhibitors, protease-activated receptor-1 antagonists, heparin antagonists, platelet-stimulating agents and thrombolytics.

The drug can be an alpha blocker. Any suitable type of alpha blocker can be used. Suitable alpha blockers include, for instance, doxazosin, prazosin and terazosin.

The drug can be a beta blocker. Any suitable type of beta blocker can be used. Suitable beta blockers include, for instance, acebutolol, atenolol, betaxolol, bisoprolol fumarate, carvedilol, esmolol, labetalol, metoprolol, nadolol, nebivolol, penbutolol, pindolol, propranolol, sotalol and timolol.

The drug can be an antihistamine. Any suitable type of antihistamine can be used. Suitable antihistamines include, for instance, azatadine, brompheniramine, cetirizine, chlorpheniramine, clemastine, cyproheptadine, desloratadine, dexchlorpheniramine, dimenhydrinate, diphenhydramine, doxylamine, fexofenadine, hydroxyzine, loratadine, phenindamine and tripelennamine.

The drug can be a steroid. Any suitable type of steroid can be used. Suitable steroids include, corticosteroids, eg. cortisone, hydrocortisone, prednisolone, methylprednisolone, triamcinolone, dexamethasone and betamethasone.

The drug can be an antiarrhythmic or cardiac stabiliser. Any suitable type of cardiac stabiliser can be used. Suitable cardiac stabilisers include, amiodarone, procainamide, and lidocaine, flecainide (Tambocor), ibutilide (Corvert), propafenone (Rythmol), quinidine, tocainide (Tonocarid).

The composition or drug delivery formulation can include one or more other active ingredients that act locally in the wound as opposed to systemically. An active ingredient, as defined herein, is a compound that provides benefit to the subject. Such active ingredients can be one or more of the drugs that were mentioned elsewhere in this specification. The active ingredient can be, for instance, an antibody, anticoagulant, antiproliferative, cytokine, cytotoxin, growth factor, interferon, haemostatic agent, hormone, lipid, demineralized bone or bone morphogenetic protein, cartilage inducing factor, oligonucleotide, polymer, polysaccharide, polypeptide, protease inhibitor, vitamin, mineral, antiseptic agent, insecticide or insect repellent, antibiotic or antifungal agent.

Any suitable amount of active ingredient can be used. In some embodiments, about 0.001 to 20 weight/volume % or weight/weight % or volume/volume % of active ingredient is used (as well as all 0.001 increments between 0.001 and 20). In some embodiments, about 0.05 to 20% weight/weight or weight/volume of active ingredient is used (as well as all 0.01 increments between 0.05 and 20). In some embodiments, about 0.1 to 10% weight/weight or weight/volume of active ingredient is used (as well as all 0.01 increments between 0.1 and 10).

For example, in some embodiments, the drug delivery formulation or composition comprises about 0.5-10 weight/volume % tetracaine (as well as all 0.01 increments between 0.5 and 10, eg. 0.51, 0.52 etc). In some embodiments, about 1-5 weight/volume % tetracaine is used. Preferably tetracaine hydrochloride is used.

For example, the composition or delivery formulation can comprise at least one antiseptic agent to, amongst other things, minimize wound contamination and infection. Any suitable type of antiseptic agent or agents can be used. Examples of suitable antiseptic agents include quaternary ammonium salts. Suitable antiseptic agents include cetrimide, povidone-iodine, chlorhexidine, iodine, benzalkonium chloride, benzoic acid, nitrofurazone, benzoyl peroxide, hydrogen peroxide, hexachlorophene, phenol, resorcinol and cetylpyridinium chloride. A preferred example is cetrimide, which is a mixture of different quaternary ammonium salts including cetrimonium bromide (CTAB).

Any suitable amount of antiseptic agent can be used. Preferably the composition comprises anywhere between approximately 0.01 weight/weight (or weight/volume or volume/volume) % and approximately 15 weight/weight (or weight/volume or volume/volume) % of antiseptic agent, which includes all 0.01 increments between 0.01 and 15%, including 0.02, 0.03 etc.

Preferably, the composition comprises approximately 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95% or 10% weight/volume cetrimide. In some embodiments, approximately 0.5% weight/volume cetrimide is used.

The term “open wound” is to be understood as a significant breach of the skin, mucous membranes and/or body cavities including a laceration, penetration, open fracture, surgical incision, ulcer, including infective or inflammatory ulcers and lesions, abrasion, or burn. Such a wound is likely to actively bleed or weep. Preferably the wound is an open skin wound.

Likewise, the term “open skin wound” is to be understood as excluding a mucous membrane wound of the alimentary and respiratory tracts and eyes, but including a skin laceration, surgical incision, ulcer, abrasion or burn and exposed underlying tissues.

According to an eighth aspect of the present invention, there is provided a method for providing pain relief to a subject having an open wound, said method comprising the step of applying topically to the significant open wound the delivery formulation according to first aspect of the invention or the composition according to the second aspect of the invention.

According to a ninth aspect of the present invention, there is provided use of the delivery formulation of the first aspect or the composition of the second aspect in the preparation of a medicament for providing pain relief to a subject having an open wound.

Depending on the form of the drug delivery formulation or composition, the composition or the delivery formulation can include one or more of the following types of ingredients: adhesive; aqueous or oily diluent; carrier; excipient; base; buffer; pH adjuster; bittering agent (i.e. foul-tasting agent); suspending agent; thickening agent; gelling agent; viscosity increasing agent; emulsifier; emollient; humectant; stabilising agent; dispersing agent/dispersant; solubiliser; skin conditioning agent; skin protectant; skin penetration enhancer; fragrance; preservative; sunscreen agent; surfactant; textural modifier; colourant; propellant, refrigerant, and, waterproofing agent.

Suitable oily or aqueous bases, carriers, diluents and excipients are inert and physiologically acceptable and include, for example: bacteriostatic saline (saline containing benzyl alcohol), cetomacrogol, cetyl alcohol, glycerine, lanolin, petrolatum based creams, gels, hydrogels, saline, short chain alcohols and glycols (e.g. ethyl alcohol and propylene glycol), and water.

Either water in oil or oil in water emulsions can be used. Examples of suitable surfactants and emulsifying agents include: non-ionic ethoxylated and non-ethoxylated surfactants, abietic acid, almond oil PEG, beeswax, butylglucoside caprate, C₁₈-C₃₆ acid glycol ester, C₉-C₁₅ alkyl phosphate, caprylic/capric triglyceride PEG-4 esters, cetomacrogol, ceteareth-7, cetereth-20, cetyl phosphate, cetyl stearyl alcohol, corn oil PEG esters, DEA-cetyl phosphate, dextrin laurate, dilaureth-7 citrate, dimyristyl phosphate, glycereth-17 cocoate, glyceryl erucate, glycerol, glyceryl laurate, G.M.S. acid stable, hydrogenated castor oil PEG esters, isosteareth-11 carboxylic acid, lecithin, lysolecithin, nonoxynol-9, octyldodeceth-20, palm glyceride, PEG diisostearate, PEG stearamine, poloxamines, polyglyceryls, potassium linoleate, PPGs, raffinose myristate, sodium caproyl lactylate, sodium caprylate, sodium cocoate, sodium isostearate, sodium tocopheryl phosphate, steareths, TEA-C₁₂-C₁₃ pareth-3 sulfate, tri-C₁₂-C₁₅ pareth-6 phosphate, and trideceths.

The composition or delivery formulation can comprise one or more types of preservative. A suitable preservative, for example, can be: benzalkonium chloride, benzoic acid, benzothonium chloride, benzyl alcohol, 2-bromo-2-nitropropane-1,3-diol, bronopol, butylated hydroxyanisole, butylated hydroxytoluene, butyl paraben, chlorophene, chlorphenesin, diazolidinyl urea, DMDM hydantoin, ethyl paraben, formaldehyde-releasing preservative, hydroquinone, iodopropynyl butylcarbamate, imidazolidinyl urea, methyldibromo glutaronitrile, methylhydroquinone, methylisothiazolinone, methyl paraben, nitrosamines, o-cymen-5-ol, phenoxyethanol, propyl paraben, quaternium-15, sodium benzoate, sodium dehydroacetate, sodium hydroxymethylglycinate, sodium metabisulfite, and sodium sulfite. Preferably, the composition includes the reducing agent such as sodium metabisulfite. Any suitable amount of sodium metabisulfite can be used, eg. up to about 3.5 mg/mL.

The composition or delivery formulation can include a colourant so that application to the wound can be verified visually. The colourant can be a pigment and/or dye. Suitable colourants include, for example, common food dyes or the ORCODERM®, ORCOBRITE® and ORCOFUR® lines of pigments and dyes sold by the Organic Dyestuffs Corporation. Preferably, the colourant is non-toxic and will not permanently stain the skin or animal hide or surrounding hair, fur or wool.

A skin conditioning agent, as defined herein, improves dry or damaged skin. Such agents, for example, include: acetyl cysteine, N-acetyl dihydrosphingosine, acrylates/behenyl acrylate/dimethicone acrylate copolymer, adenosine, adenosine cyclic phosphate, adensosine phosphate, adenosine triphosphate, alanine, albumen, algae extract, allantoin and deriviatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, chamomilla recutita (matricaria) flower extract, cholecalciferol, cholesteryl esters, coco-betaine, coenzyme A, corn starch modified, crystallins, cycloethoxymethicone, cysteine DNA, cytochrome C, darutoside, dextran sulfate, dimethicone copolyols, dimethylsilanol hyaluronate, DNA, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, kinetin, lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanol, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sorbitol, sphingolipids, stearamidopropyl betaine, stearyl palmitate, tocopherol, tocopheryl acetate, tocopheryl linoleate, ubiquinone, Vitis vinifera (grape) seed oil, wheat amino acids, xanthan gum, and zinc gluconate.

The composition or delivery formulation can include a skin penetration enhancer for enhancing the penetration of active ingredients, such as the anaesthetic agent or the analgesic agent. Any suitable type of enhancer can be used. Examples of suitable enhancers may include solvents, detergents or low carbon alcohols such as dimethylsulfoxide, oleyl alcohol, propylene glycol, methyl pyrrolidone and dodecylazyl cycloheptan 2-one.

The drug delivery formulation or composition can comprise one or more of the following adhesives, thickening agents, gelling agents and/or viscosity increasing agents: acrylamides copolymer, agarose, amylopectin, calcium alginate, calcium carboxymethyl cellulose, carbomer, carboxymethyl chitin, castor oil derivatives, cellulose gum, cellulosic preparation, cetyl alcohol, cetostearyl alcohol, dextrin, gelatin, hydroxy cellulose, hydroxyethylcellulose, hydroxypropylcellulose, hydroxpropyl starch, inert sugar, magnesium alginate, methylcellulose, microcrystalline cellulose, pectin, PEG's, polyacrylic acid, polymethacrylic acid, polyvinyl alcohol, quaternium ammonium compound of bentonite or zinc stearate, sorbitol, PPG's, sodium acrylates copolymer, sodium carrageenan, xanthum gum, and yeast beta-glucan.

The composition can comprise an insecticide or insect repellent to stop insects from infesting a wound of the subject. Any suitable type of insecticide or insect repellent can be used. Examples of suitable insecticides include: trichlorfon, triflumeron, fenthion, bendiocarb, cyromazine, dislubenzuron, dicyclanil, fluazuron, amitraz, deltamethrin, cypermethrin, chlorfenbinphos, flumethrin, ivermectin, abermectin, avermectin, doramectin, moxidectin, zeti-cypermethrin, diazinon, spinosad, imidacloprid, nitenpyran, pyriproxysen, sipronil, cythioate, lufenuron, selamectin, milbemycin oxime, chlorpyrifos, coumaphos, propetamphos, alpha-cypermethrin, high cis cypermethrin, ivermectin, diflubenzuron, cyclodiene, carbamate and benzoyl urea.

The subject can be a human. The subject can be another type of mammal or animal. The subject can be a farm animal or livestock, such as a sheep, horse, cow, goat or pig. The subject can be a companion animal, such as a cat or dog. The subject can be a laboratory animal, such as a mouse, rat or rabbit. Preferably the subject is human or dog, pig, piglet, horse, lamb or calf.

The delivery formulation or composition can be used for an animal husbandry procedure. The procedure can be, for example, castration, mulesing, shearing, tail docking, ear tagging, de-horning, branding or marking.

The delivery formulation or composition can be applied to the open wound in any suitable form. The composition can be applied to the wound in a liquid form or other free-flowing form. The composition can be applied to the wound as a spray-on liquid or spray-on gel so as to minimise pain related to touching or handling a wound (caused by castration, for example), minimise the risk of infection from skin contamination and so that the wound need not be disturbed more than necessary. Alternatively, the delivery formulation or composition can be applied as a gel by hand, or squeezed from a tube to fill a wound caused, say, during a castration or de-horning procedure.

Alternatively, the delivery formulation or composition can be inserted into the wound as a dissolvable capsule or tablet.

Once applied to the significant open wound, the delivery formulation or composition can be, for example, in form of an adhesive/sticky/tenacious ointment, gel, lotion, crème, cream, emulsion, paste, solution or suspension, or may set and/or form a physical barrier, ‘skin’ or film. The delivery formulation or composition can be in a sustained-release form, whereby the drug/s or active/s are slowly released to provide a therapeutic effect over an extended period of time. The delivery formulation or composition can be incorporated into a bandage, plaster, dressing, wipe or tissue.

The delivery formulation or composition can be applied as a metered dose.

According to a tenth aspect of the present invention, there is provided a method for administering pain relief to a large number of animals for a husbandry procedure in a short period of time, said method comprising the steps of:
creating an open wound on each said animal in accordance with the husbandry procedure; and
applying to the wound of each said animal the drug delivery formulation of the first aspect of the invention or the drug delivery composition of the second aspect of the invention to provide a therapeutic effect for a predetermined period of time.

The animal husbandry procedure is preferably selected from castration, mulesing, shearing, tail docking, ear tagging, de-horning, branding and marking.

Such a method allows for the high throughput of animals, with minimal stress and handling due to the unique properties of the composition; namely, it can be applied topically, rather than needle-injected.

The drug delivery formulation or composition can form or can be in the form of an adhesive gel after being applied to the significant open wound. The drug delivery formulation or composition can comprise a hydrophilic or hydroalcoholic gelling agent. The composition or delivery system can comprise about 1 to 20 g per litre of at least one type of gum or cellulosic preparation, including all about 0.1 g increments between 1 and 20, including 1.1, 1.2 etc. The composition or delivery system can comprise a polyhydric alcohol in combination with a cellulosic preparation; for example, hydroxy cellulose (eg. hydroxyethyl cellulose, ethylhydroxy cellulose) in combination with non-crystallising liquid sorbitol. The composition or delivery formulation can comprise about 5 mg/mL hydroxy cellulose (eg. hydroxyethyl cellulose) in combination with about 100 mg/mL non-crystallising liquid sorbitol (70%).

Preferably, the composition or delivery formulation is in the form of a liquid prior to having been applied to a wound. Preferably, the composition or delivery formulation forms, or is in the form of, a sticky, viscous, adhesive solution or gel when applied to a wound. Preferably, the composition or delivery formulation is in the form of a spray-on gel that can coat the wound of the subject and can maximise delivery of the drug or drugs to the wound by way of staying moist and viscous (i.e. “sticky”).

Preferably, the composition or delivery formulation forms an effective long-lasting barrier over the wound. The term “long-lasting barrier” is to be understood as meaning a barrier/seal that is substantially capable of remaining intact over a wound for hours, days, a week or even weeks, or until the wound has naturally sealed or the pain has otherwise abated by way of the natural healing process—eg. about 1, 2, 3, 4, 5, 6, 7, 8, 9, 10, 11, 12, 13, 14, 15, 16, 17, 18, 19, 20, 21, 22, 23 or 24 hours, or about one, two, three, four, five, six or seven days, or one, two, three, four or more weeks.

The barrier preferably aids in the healing process, presumably by minimising or preventing water loss from the wound and by acting as a barrier against microbial contamination.

Preferably the composition or delivery formulation is in the form of a liquid that thickens to an adhesive gel when reacting with physiological fluids of the open wound. Preferably, the composition or delivery formulation enables systemic absorption of the drug/s in a predictable manner, at a predictable rate.

The term “providing a therapeutic effect for a predetermined period of time” is to be understood as meaning about 1, 2, 3, 4, 5, 6, 7, 8, 9, 10, 11, 12, 13, 14, 15, 16, 17, 18, 19, 20, 21, 22, 23 or 24 hours, or about one, two, three, four, five, six or seven days, or one, two, three, four or more weeks, or until the wound has naturally sealed or the pain has otherwise abated by way of the natural healing process.

Preferably the composition or delivery formulation is capable of being applied directly to a significant open wound.

Preferably the composition or delivery formulation is capable of coating and adhering to the wound.

Preferably the composition or delivery formulation is capable of controlled and/or prolonged release of at least one drug (and other active ingredient if present) that acts systemically or locally and systemically.

Preferably the composition or delivery formulation is biocompatible and absorbable such that they do not require removal (ie. “set and forget”).

Preferably the composition or delivery formulation comprises an adhesive gel base capable of coating and adhering to a significant open wound and providing prolonged contact with the wound and controlled and/or prolonged release of the at least one drug (and active ingredient if present).

The composition or delivery formulation is preferably used for surgical wounds, traumatic wounds, infective lesions or chronic wounds.

The composition or delivery formulation is preferably used for piglet castration or otherwise in the livestock husbandry.

In some embodiments, the least one drug is an analgesic, NSAID, anaesthetic, antibiotic, antifungal or antihelminthic.

In some embodiments, the at least one drug is an analgesic agent, such as meloxicam.

In some embodiments, the at least one active ingredient is a local anaesthetic, such as lidocaine, which has local and central effects.

In some embodiments, the composition is applied as a liquid to an open wound, such as a spray-on liquid.

In some embodiments, the drug delivery formulation or composition forms an adhesive gel when applied to an open wound.

In some embodiments, the drug delivery formulation or composition forms a long-lasting barrier over an open wound.

In some embodiments, the at least one drug is substantially uniformly dispersed throughout the drug delivery formulation or composition.

In some embodiments, the at least one drug is released in a controlled or predictable manner from the drug delivery formulation or composition such as to deliver safe therapeutic systemic drug levels.

In some embodiments, the at least one active ingredient is an antiseptic, such as cetrimide.

In some embodiments, the composition comprises an antiseptic, such as cetrimide.

In some embodiments, the drug delivery formulation or composition comprises a reducing agent or preservative, such as sodium metabisulfite.

In some embodiments, the drug delivery formulation comprises a gelling agent or thickener, such as hydroxyethyl cellulose.

In some embodiments, the drug delivery formulation comprises a gelling agent or thickener, such as non-crystallising liquid sorbitol (70%).

In some embodiments, the drug delivery formulation or composition comprises a colourant, such as a dye.

In some embodiments, the drug delivery formulation or composition comprises a pH adjuster or buffering agent such as monoethanolamine, citric acid or disodium hydrogen orthophosphate.

In some embodiments, the drug delivery formulation or composition comprises a stabilising or suspending agent, such as microcrystalline cellulose and/or sodium carboxymethylcellulose.

In some embodiments, the drug delivery formulation or composition comprises a dispersant, such as glycerine.

In some embodiments, the drug delivery formulation or composition comprises an emulsifier or solubiliser, such as polyoxyl castor oil.

The topical drug delivery composition can comprise a refrigerant. ‘Refrigerant’ as used herein is a volatile liquid that evaporates on contact with the wound and/or a pressurised gas that when contacting the wound causes a local refrigerant effect whereby the wound is cooled, chilled or frozen. In this way, the refrigerant can provide local anaesthesia, such as for burns, incisions and other wound types caused by surgical and animal husbandry procedures. Rapid evaporation of the volatile liquid from the wound or cold gas striking the wound's surface causes a drop in temperature and results in temporary interruption of pain sensation.

The topical drug delivery composition can comprise any suitable type of refrigerant. The topical drug delivery composition can comprise one type of refrigerant or more than one type of refrigerant. The refrigerant can be a gas. The refrigerant can be a volatile liquid. The at least one refrigerant can be flammable or non-flammable. The topical drug delivery composition can comprise 1, 2, 3, 4, 5 or even more types of refrigerants. In some embodiments, the topical drug delivery composition can comprise a blend or mixture of two or more refrigerants. In some embodiments, the 2 or more refrigerants can either be a combination of gas and gas, volatile liquid and gas, or volatile liquid and volatile liquid.

Examples of suitable refrigerants include any one or more of the following:
a compressed gas such as an inert gas, such as nitrogen, carbon dioxide, nitrous oxide, oxygen or air;
a liquefied hydrocarbon such as methane, ethane, ethyl alcohol, propane, butane, n-butane, isobutane, pentane, isopentane, n-pentane; a mixture of 2, 3, 4 or more hydrocarbons (eg. a mixture of n-butane, isobutane and propane, or a mixture of propane and butane);
a fluorinated hydrocarbon such as trichloromonofluromethane, dichlorodifluoromethane, dichlorotetrafluroethane, 1,1,1,3,3 pentafluoropropane or 1,1,1,2 Tetrafluoroethane; liquid nitrogen;
an ether such as dimethyl ether (DME) or methyl ethyl ether; or
a hydrofluoroalkane (HFA) such as HFA 134a (1,1,1,2,-tetrafluoroethane) or HFA 227 (1,1,1,2,3,3,3-heptafluoropropane), or a combination of these.

The topical drug delivery composition can comprise any suitable amount of refrigerant. Preferably the topical drug delivery composition comprises anywhere between approximately 10 and approximately 99.9 weight/weight (or weight/volume or volume/volume) % of refrigerant, which includes all 0.1 increments between 10 and 99.5%, including 10.5, 11, 11.5.

In some embodiments the topical drug delivery composition comprises between approximately 20% weight/weight and 80% weight/weight refrigerant. In some embodiments the topical drug delivery composition comprises between approximately 30% weight/weight and approximately 70% weight/weight refrigerant. In some embodiments, the topical drug delivery composition comprises approximately 30%, 35%, 40%, 45%, 50%, 55%, 60%, 65%, 70%, 75% or 80% weight/weight refrigerant. More preferably, the topical drug delivery composition comprises approximately 30%, 35%, 40%, 45%, 50%, 55%, 60%, 65%, 70%, 75% or 80% weight/weight hydrocarbon/s or ether/s.

The topical drug delivery composition can be in the form of a sprayable stream, sprayable mist or sprayable foam. The topical drug delivery composition can comprise or can be delivered from a pressurised spray container or can, in which case it may contain at least one propellant. In some embodiments the at least one refrigerant can function as the at least one propellant. The topical drug delivery composition can further comprise at least one solvent for the propellant, but this will depend on the nature of the propellant.

In some embodiments the topical drug delivery composition comprises a gaseous suspension of liquid particles. In some embodiments the topical drug delivery composition comprises an aerosol mist comprising liquid particles. In some embodiments the topical drug delivery composition comprises a foam, whereby the foam comprises gas pockets trapped in liquid.

Most preferably, the topical drug delivery composition is in the form of a sprayable foam. In some embodiments the topical drug delivery composition is in the form of a liquid that is expelled from the pressurised container as a foam and sets as a sticky viscous gel when exposed to the wound, after the refrigerant evaporates or otherwise dissipates.

The topical drug delivery composition can comprise a delivery nozzle, cap, tip or actuator.

Any suitable type of propellant or blend of propellants can be used. The propellant or propellant blend can be flammable or non-flammable. The propellant can be a compressed gas, soluble gas or liquefied gas. The propellant can also act as solvent, diluent, viscosity modifier or freezant.

Examples of suitable propellants include any one or more of the following:
a compressed gas such as an inert gas, such as nitrogen, carbon dioxide, nitrous oxide, oxygen or air;
a liquefied hydrocarbon such as methane, ethane, ethyl alcohol, propane, butane, n-butane, isobutane, pentane, isopentane, n-pentane; a mixture of 2, 3, 4 or more hydrocarbons (eg. a mixture of n-butane, isobutane and propane, or a mixture of propane and butane);
a fluorinated hydrocarbon such as trichloromonofluromethane, dichlorodifluoromethane, dichlorotetrafluroethane, 1,1,1,3,3 pentafluoropropane or 1,1,1,2 Tetrafluoroethane;
liquid nitrogen;
an ether such as dimethyl ether (DME) or methyl ethyl ether; or
a hydrofluoroalkane (HFA) such as HFA 134a (1,1,1,2,-tetrafluoroethane) or HFA 227 (1,1,1,2,3,3,3-heptafluoropropane); or a combination of these.

The topical drug delivery composition can comprise any suitable amount of propellant. Preferably the topical drug delivery composition comprises anywhere between approximately 10 and approximately 99.9 weight/weight (or weight/volume or volume/volume) % of propellant, which includes all 0.1 increments between 10 and 99.5%, including 10.5, 11, 11.5 etc.

In some preferred embodiments, the refrigerant is carbon dioxide or other type of compressed gas, which also functions as the propellant. In some preferred embodiments refrigerant and/or propellant is used to balance in an aerosol container, particularly when a compressed gas.

The topical drug delivery composition can be applied for any suitable period of time. The time period will typically be between about 1 and 10 seconds, although it may be shorter or longer (eg. up to 15, 20, 25 or 30 seconds). Preferable application times include, but are not limited to, 1, 1.5, 2, 2.5, 3, 3.5, 4, 4.5, 5, 5.5, 6, 6.5, 7, 7.5, 8, 8.5, 9, 9.5 and 10 seconds.

The topical drug delivery composition can either cool, chill or freeze the wound, but preferably cools it to a temperature from between about −20° C. up to about 10° C., including −20, −19.5, −19 . . . etc . . . 0, 0.5, 1, 1.5, 2, 2.5, 3 . . . etc . . . 7, 7.5, 8, 8.5, 9, 9.5 and 10° C. In some embodiments the topical drug delivery composition can cool the wound to a temperature of between about 1 and 2° C. In some embodiments the topical drug delivery composition can cool the wound to a temperature below about 9° C. or 10° C.

In a preferred embodiment (‘Formulation 1’) the composition comprises:

| Ingredient | Approximate % w/v |
|---|---|
| Hydroxyethyl cellulose | 0.5 |
| Sodium metabisulfite | 0.15 |
| Cetrimide | 0.5 |
| Meloxicam | 0.1 |
| Monoethanolamine | Quantity to suit to adjust pH |
| Sorbitol 70% AU | 10 |
| Dye (optional) | Quantity to suit |
| Water | Quantity to suit |
| Approximate pH 8.77 | |

In a preferred embodiment (‘Formulation 2’) the composition comprises:

| Ingredient | Approximate % w/v |
|---|---|
| Hydroxyethyl cellulose | 0.5 |
| Polyoxyl castor oil | 1 |
| Tetracaine HCl | 2 |
| Sodium metabisulfite | 0.15 |
| Cetrimide | 0.5 |
| Monoethanolamine | Quantity to suit |
| Meloxicam | 0.1 |
| Sorbitol 70% AU | 10 |
| Dye (optional) | Quantity to suit |
| Water | Quantity to suit |
| Approximate pH 5.78 | |

In a preferred embodiment (‘Formulation 3’) the composition comprises:

| Ingredient | Approximate % w/v |
|---|---|
| Cetrimide | 0.02 |
| Hydroxyethyl cellulose | 0.5 |
| Polyoxyl castor oil | 1 |
| Tetracaine HCl | 5 |
| Sodium metabisulfite | 0.15 |
| Cetrimide | 0.48 |
| Meloxicam | 0.1 |
| Sorbitol 70% AU | 10 |
| Monoethanolamine | Quantity to suit |
| Citric acid | Quantity to suit |
| Dye (optional) | Quantity to suit |
| Water | Quantity to suit |
| Approximate pH 5.74 | |

In a preferred embodiment (‘Formulation 4’) the composition comprises:

| Ingredient | Approximate % w/v |
|---|---|
| Microcrystalline Cellulose and | 0.2 |
| Sodium Carboxymethylcellulose | |
| Cetrimide | 0.50 |
| Hydoxyethyl cellulose | 0.5 |
| Citric acid | Quantity to suit |
| Disodium hydrogen | Quantity to suit |
| orthophosphate•2H₂0 | |
| Tetracaine HCl | 2 |
| Sodium metabisulfite | 0.15 |
| Glycerine | 4 |
| Meloxicam | 0.1 |
| Sorbitol 70% AU | 4 |
| Dye (optional) | Quantity to suit |
| Water | Quantity to suit |
| Approximate pH 3.20 | |

In a preferred embodiment (‘Formulation 5’) the composition comprises:

| Ingredient | Approximate % w/v |
|---|---|
| Microcrystalline Cellulose and | 0.2 |
| Sodium Carboxymethylcellulose | |
| Cetrimide | 0.50 |
| Hydoxyethyl cellulose | 0.5 |
| Citric acid | Quantity to suit |
| Disodium hydrogen | Quantity to suit |
| orthophosphate•2H₂0 | |
| Tetracaine HCl | 5 |
| Sodium metabisulfite | 0.15 |
| Glycerine | 4 |
| Meloxicam | 0.1 |
| Sorbitol 70% AU | 4 |
| Dye (optional) | 0.005 |
| Water | Quantity to suit |
| Approximate pH 3.18 | |

Any drug that is currently formulated for injection can theoretically be delivered via an open wound using the drug delivery formulation. Drugs for injection are usually water based fluids—eg lidocaine, morphine and gentamicin.

Preferably the drug delivery formulation or composition (like Tri-Solfen™) can provide slow/prolonged release. They can give a slower/lower more prolonged peak effect.

In some embodiments, drug delivery formulations or compositions can be designed for application to wounds for systemic drug delivery other than in fluid form—eg pre-soaked gauzes, or dose determined dissolvable discs, gel-caps, microspheres or “patches” applied to wounds.

In some embodiments, the drug delivery formulation or composition can be a variant of a Tri-solfen™ base formulation (note—this can include examples with and without cetrimide).

Single systemic active delivery examples follow:
1a) lidocaine 2-5% (20-50 mg/ml)—(may be with or without adrenalin 1:2000-1:200,000).
1b) morphine 0.05-1% (0.5-10 mg/ml)—or equivalent dose of other opioid (may be with or without adrenalin 1:2000-1:200,000).
1c) meloxicam 0.1-1% (1 mg-10 mg/ml)—or equivalent dose of other NSAID (may only be without adrenalin).
1d) tetanus toxoid 10 fl (flocculation units)/ml.
1e) tetanus immunoglobin 50-500 IU/ml.
1f) penicillin 60 mg-1.2 g/ml, cefoxitin 1-10 g/ml, vancomycin 30 mg-1 g/ml, clindamycin 60-600 mg/ml or gentamicin 1-40 mg/ml.
1g) tranexamic acid 100 mg/ml (systemic haemostatic agent).
1f) adrenalin 0.1-1 mg/ml.
1g) hydrocortisone 1-100 mg/ml.

Multiple systemic active delivery examples:
may contain any combination of the above (with or without cetrimide or adrenalin as above)—e.g.:
lidocaine, plus morphine/, or lidocaine plus tetanus toxoid.
lidocaine, plus meloxicam plus tetanus toxoid/or lidocaine, plus meloxicam, plus gentamicin/or lidocaine, plus morphine, plus tranexamic acid.
lidocaine plus morphine, plus tranexamic acid plus clindamycin.
Local and systemic delivery. May contain any of 1a-1-g above with:
bupivacaine 0.25-0.75%.
tetracaine ˜1-10%.
alternative antiseptic—eg chlorhexidine 0.05-0.5%.
alternative (locally acting) haemostatic agent eg topical fibrin.

In some embodiments, the drug delivery formulation or composition can be a base formulation different from Tri-solfen™. This is for any example containing meloxicam and adrenalin, as this requires a different base due to pH issues.

In a preferred embodiment (‘Formulation 6’) the composition comprises:

| Ingredient | Approximate % w/v |
|---|---|
| Part A | |
| Water | 40 |
| Microcrystalline Cellulose and | 0.2 |
| Sodium Carboxymethylcellulose | |
| Cetrimide (99.28*(100 − 0.15/100) | 0.02 |
| (=99.13% w/w as is) | |
| Hydoxyethyl cellulose | 0.5 |
| Part B | |
| Water | 40 |
| Citric acid | 1.5 |
| Disodium hydrogen orthophosphate•2H₂0 | 5 |
| Tetracaine HCl*purity (99.3% w/w as is) | 2 |
| Sodium metabisulfite | 0.15 |
| Cetrimide (99.28*(100 − 0.15/100) | 0.48 |
| (=99.13% w/w as is) | |
| Adrenaline acid tartrate*purity | 0.00495 |
| (99.0*(100 − 0.21/100) = 98.79% w/w as is) | |
| Part C | |
| Glycerine | 4 |
| Meloxicam | 0.1 |
| (Neosorb 70/20) Sorbitol 70% AU | 4 |
| Part D | |
| Dye (optional) | 0.005 |
| Water (Part B) | 6 |
| Approximate pH 3.16 | |

In a preferred embodiment (‘Formulation 7’) the composition comprises:

| Ingredient | Approximate % w/v |
|---|---|
| Part A | |
| Water | 40 |
| Microcrystalline Cellulose and | 0.2 |
| Sodium Carboxymethylcellulose | |
| Cetrimide (99.28*(100 − 0.15/100) | 0.02 |
| (=99.13% w/w as is) | |
| Hydoxyethyl cellulose | 0.5 |
| Part B | |
| Water | 40 |
| Citric acid | 1.0 |
| Disodium hydrogen orthophosphate•2H₂0 | 1 |
| Tetracaine HCl*purity (99.3% w/w as is) | 2 |
| Sodium metabisulfite | 0.15 |
| Cetrimide (99.28*(100 − 0.15/100) = | 0.48 |
| 99.13% w/w as is) | |
| Adrenaline acid tartrate*purity | 0.00495 |
| (99.0*(100 − 0.21/100) = 98.79% w/w as is) | |
| Part C | |
| Glycerine | 4 |
| Meloxicam | 0.1 |
| (Neosorb 70/20) Sorbitol 70% AU | 4 |
| Part D | |
| Dye (optional) | 0.005 |
| Water (Part B) | 6 |
| Approximate pH 3.55 | |

Single systemic active delivery (with adrenalin) examples—may be with or without cetrimide.

2a) meloxicam 0.1-1% (1 mg-10 mg/ml)—or equivalent dose of other basic NSAID—with adrenalin 1:2000-1:200,000.

Multiple drugs and/or active ingredients.
2b) meloxicam plus tetracaine plus adrenalin—doses as above.
2c) meloxicam plus lidocaine plus adrenalin—doses as above.
2d) meloxicam plus lidocaine plus bupivacaine plus adrenalin—doses as above.

Having broadly described the invention in its various embodiments, trials leading to and providing confirmation of the inventive concept, with non-limiting examples of embodiments will now be given.

### BRIEF DESCRIPTION OF FIGURES

FIG. 1 is a graphic representation of lidocaine and bupivacaine plasma levels in neonatal piglets treated with Tri-Solfen topical application to the castration wound.

FIG. 2 is a graphic representation of bupivacaine levels in piglets treated with either Tri-Solfen (TS) or bupivacaine for injection (BUP) topical application to the castration wound.

FIG. 3 is a graphical representation of tetracaine levels in plasma of tetracaine treated piglets.

FIG. 4 is a graphical representation of meloxicam levels in plasma of meloxicam treated piglets.

FIG. 5 is a graphical representation of meloxicam levels in plasma of tetracaine and meloxicam treated piglets.

FIG. 6 is graphic representation of motor response scores during castration in piglets treated with tetracaine, meloxicam, or a combination as compared with placebo, applied topically to the open wound prior to severing the cord.

FIG. 7 is graphic representation of motor response scores of piglets from 1 minute up to 12 hours following castration in piglets treated with tetracaine, meloxicam, or a combination as compared with placebo, applied topically to the castration wound.

### BEST MODES FOR CARRYING OUT THE INVENTION

### Tri-Solfen™ Trials Leading to the Inventive Concept

In the inventor's earlier piglet trials with the spray-on anaesthetic and analgesic product Tri-Solfen™, she incorporated adrenalin in the product so as to minimise systemic absorption and to prevent toxicity, because the prevailing wisdom was that there would be unpredictable high-level absorption of the anaesthetic agents (lidocaine/lignocaine and bupivacaine) through open wounds that could be dangerous. Consequently, due to the inclusion of adrenaline, the inventor expected to see minimal level of systemic absorption of the anaesthetic agents as had been evident in preceding trials involving applications to disbudding wounds in cattle.

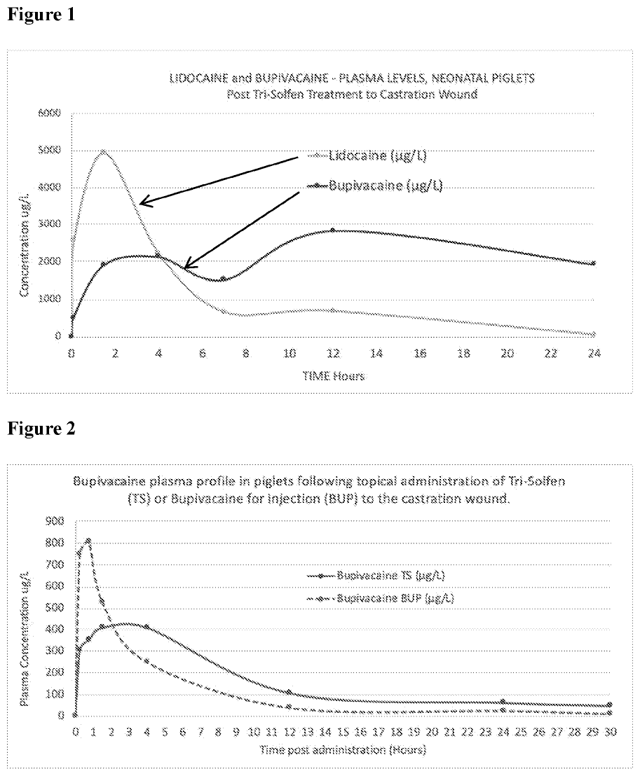

To the inventor's surprise (being a cardiologist), however, lidocaine/lignocaine levels in neonatal piglets treated via the castration wound although not toxic, were in the range that was considered therapeutic (for cardiac arrhythmia prevention or central analgesic effects 1000-5000 μg/L) and remained at those levels for a relatively prolonged period (5 hrs for lidocaine/lignocaine and 24 hrs for bupivacaine—see FIG. 1). Although this was not the intended effect, this struck the inventor at the time how hard it was to achieve lidocaine levels in the therapeutic range and keep them there for longer than an hour by any other method (unless using an IV infusion).

There was a surprising consistency between piglets treated with the same product as well, rather than wide variety and unpredictability which was considered the norm. In retrospect, this was possibly because the inventor was in the unusual situation of examining a large number of wounds of the same type at the same time, which situation would be unusual in most hospital or clinical situations where wounds may be more variable. Variability of wound size and types in previous trials, as well as differences in the base formulations of products used, may explain why wound absorption has previously been considered impractical, inconsistent, unpredictable and unsafe as a method of systemic drug delivery and why no one has ever considered that there may be situations where it is possible—and where methods/products could be developed to achieve it.

Also, unexpectedly, the inventor discovered that there are different drug product profiles of absorption via wounds depending on the nature of the delivery formulations that make some more suitable and others less suitable for different wound situations. The plasma profiles of product delivered in a viscous gel base with adrenalin (Tri-Solfen™) indicated a “midway” speed of absorption profile that was faster than oral or IM absorption, but slower and longer lasting than IV. When the same dose of a drug was delivered by the same route, but in a water-based solution (i.e. a solution for IV injection) the absorption profile showed a rapid and higher peak with more rapid depletion, more characteristic of intravenous injection (FIG. 2).

This gave the inventor a thought that products could potentially be specifically designed for wound administration as a novel means of achieving relatively rapid onset, prolonged effect systemic administration of drugs to therapeutic levels, that is faster onset than oral or IM, and less risky and longer lasting than IV—which is of course perfect for wound pain relief such as with NSAIDs—that have central as well as local effect.

Up until now people have thought of administering drugs for systemic effect via oral, buccal, rectal, s.c, IM, IV, intra-peritoneal etc. However as far as the inventor is aware, no one has thought of inventing or developing products for intended systemic drug delivery via topical application to open wounds—or to achieve a combination of local and systemic effects with the one open wound application.

### Example 1—Study to Determine the Porcine Plasma Profiles of Tetracaine and Meloxicam when Each is Applied Topically, Independently or in Combination to the Surgical Site of Piglets Undergoing Castration

### Background

Meloxicam is a long acting NSAID that has proven efficacy to reduce pain related behaviour in piglets from 2-4 hours post castration (delivered by IM injection at a dose of 0.4 mg/kg 20-30 minutes prior to the procedure). Tetracaine is a rapid onset surface anaesthetic, that is rapidly metabolised by plasma-esterases, such that may induce high potency local effects with minimal systemic effects. In-house trials, had proven tetracaine effective to ameliorate pain during castration and in the first minutes and 1-2 hours following the procedure when applied directly to the open castration wound, 20 seconds prior to severing the cordal tissues, at doses of 1 ml/kg of 2% or 5% solution. Topical wound application formulations were thus developed to examine the potential for systemic NSAID analgesic drug (meloxicam) delivery via topical wound application, applied alone, or combined with topical local anaesthetic (tetracaine) to the castration wound in piglets. Topical wound application formulations were developed containing meloxicam alone, tetracaine alone and combinations of meloxicam and tetracaine to examine systemic absorption and clinical effects.

Study 1: The aim of this study was to determine the porcine plasma profiles of tetracaine and meloxicam when each is applied topically, independently or in combination to the surgical site of piglets undergoing castration.

### Allocation

Litters containing male piglets considered suitable for enrolment into the study were selected. Individual males were identified (uniquely numbered ear tag).

### Investigational Veterinary Products (IVPs).

Investigational formulations were developed based on the Tri-Solfen™ base delivery formulation (viscous gel, known to be safe for wound application). Modifications were made as required to include actives meloxicam and/or tetracaine) and to promote systemic absorption of meloxicam.

TABLE 1
Solution 5% Tetracaine and 0.5% Cetrimide
Name: Solution 5% Tetracaine and 0.5% Cetrimide
Composition: 50 mg/mL Tetracaine HCl; 5 mg/mL Cetrimide
Dose Level: See Table 4

| No. | Material | % w/v | g/500 mL |
|---|---|---|---|
| 1 | RO water (Part A) * adjusted for active | 80.99 | 404.95 |
| | purity | | |
| 2 | Natrosol 250 HHR Hydoxyethyl | 0.5 | 2.5 |
| | cellulose | | |
| 3 | Tetracaine HCl*purity (99.3% w/w as | 5.0 | 25 |
| | is) | | |
| 4 | Sodium metabisulfite | 0.15 | 0.75 |
| 5 | Cetrimide*purity (Part A) | 0.02 | 0.1 |
| | (99.28*(100 − 0.15/100) = 99.13% w/w | | |
| | as is) | | |
| 6 | Cetrimide*purity (Part B) | 0.48 | 2.4 |
| | (99.28*(100 − 0.15/100) = 99.13% w/w | | |
| | as is) | | |
| 8 | Neosorb 70/20/Sorbitol 70% AU | 10 | 50 |
| 9 | Dye FD&C Blue No. 1 | 0.005 | 0.025 |
| 10 | RO Water (Part B) | 5.84 | 29.2 |

| Test Descriptor | Results | Units |
|---|---|---|
| Appearance | Clear blue solution | |
| Specific gravity [20° C.] | 1.036 | |
| Viscosity [DVI Sp3, | 244 | cP |
| 100 RPM, 20° C.] | | |
| pH | 3.15 | |
| Tetracaine hydrochloride | 50.0 | mg/mL |
| Cetrimide | 5.33 | mg/mL |

TABLE 2
Solution 0.1% Meloxicam and 0.5% Cetrimide
Name: Solution 0.1% Meloxicam and 0.5% Cetrimide
Composition: 1.0 mg/mL Meloxicam; 5.0 mg/mL Cetrimide
Dose Level: See Table 4

| No. | Material | % w/v | g/500 mL |
|---|---|---|---|
| 1 | RO water | 40 | 400 |
| 2 | Cetrimide (99.28*(100 − 0.15/100) = | 0.02 | 0.1 |
| | 99.13% w/w as is) | | |
| 3 | Natrosol 250 HHR | 0.5 | 2.5 |
| | Part B | | |
| 4 | Sodium metabisulfite | 0.15 | 0.75 |
| 5 | Cetrimide (99.28*(100 − 0.15/100) = | 0.48 | 2.4 |
| | 99.13% w/w as is) | | |
| 6 | Meloxicam | 0.1 | 0.5 |
| 7 | Monoethanolamine | Adjust pH to | |
| | | clear solution | |
| 8 | Neosorb 70/20 Sorbitol 70% AU | 10 | 50 |
| | Part C | | |
| 9 | Dye FD&C Blue No. 1 | 0.005 | 0.025 |
| 10 | RO Water (Part B) | 12 | 60 |

| Item | Test Descriptor | Results | Units |
|---|---|---|---|
| 1 | Appearance | Clear purple solution | |
| 2 | Viscosity [DVI Sp3, 100 RPM, | 163 | cP |
| | 20° C.] | | |
| 3 | pH | 8.77 | |
| 4 | Specific gravity [20° C.] | 1.036 | |
| 5 | Cetrimide | 5.13 | mg/mL |
| 6 | Meloxicam | 0.98 | mg/mL |

TABLE 3
Solution 5% Tetracaine; 0.5% Cetrimide and 0.1% Meloxicam
Name: Solution 5% Tetracaine; 0.5% Cetrimide and 0.1% Meloxicam
Composition: 50 mg/mL Tetracaine HCl;
5 mg/mL Cetrimide; 1 mg/mL Meloxicam
Dose Level: See Table 4

| No. | Material | % w/v | g/500 mL |
|---|---|---|---|
| 1 | RO water | 40 | 400 |
| 2 | Cetrimide (99.28*(100 − 0.15/100) = | 0.02 | 0.1 |
| | 99.13% w/w as is) | | |
| 3 | Natrosol 250 HHR | 0.5 | 2.5 |
| | Part B | | |
| 4 | Polyoxyl castor oil, Kolliphor EL | 1 | 5.0 |
| 5 | Tetracaine HCl*purity (99.3% w/w as | 5 | 25.2 |
| | is) | | |
| 6 | Sodium metabisulfite | 0.15 | 0.75 |
| 7 | Cetrimide (99.28*(100 − 0.15/100) = | 10.48 | 2.4 |
| | 99.13% w/w as is) | | |
| 8 | Monoethanolamine | Raise pH to | 0.5 mL + |
| | | 6.4 ± 0.1 | 2 drops |
| 9 | Meloxicam | 0.1 | 0.5 |
| 10 | Neosorb 70/20 Sorbitol 70% AU | 10 | 50 |
| | Part D | | |
| 11 | Dye FD&C Blue No. 1 | 0.005 | 0.025 |
| 12 | RO Water (Part B) | 6 | 30 |

| Item | Test Descriptor | Results | Units |
|---|---|---|---|
| 1 | Appearance | Clear, green-blue solution | |
| 2 | Specific gravity [20° C.] | 1.037 | |
| 3 | Viscosity [DVI Sp3, | 242 | cP |
| | 100 RPM, 20° C.] | | |
| 4 | pH | 5.78 | |
| 5 | Tetracaine | 49.9 | mg/mL |
| | hydrochloride | | |
| 6 | Cetrimide | 5.06 | mg/mL |
| 7 | Meloxicam | 0.98 | mg/mL |

Source & Storage: The IVP was sourced and transported to the animal phase site by the Sponsor and stored at ambient conditions.

### Treatment

Dose Calculation, Preparation & Administration: Study animals were dosed according to the Treatment Regime outlined in Table 4 below. No preparation of any of the IVPs was performed other than inversion of each container 2-3 times prior to treatment. The IVPs were administered using 1 mL [Tri-Solfen™] applicators each with a ball-point injection tip fitted to allow IVP deposition into the scrotal sac.

Study animals were observed in a group setting at each blood collection timepoint.

TABLE 4
Treatment Regime

| | | | | Dose | |
|---|---|---|---|---|---|
| | | | | Adminis- | |
| Animal | | Bodyweight | | tered | |
| ID | Group | (Day 0) (kg) | Treatment | (mL) | Time |
| 1 | 1 | 2.19 | Tetracaine | 2.0 | 8:12:00 AM |
| 2 | | 2.65 | 5% | 2.0 | 8:14:00 AM |
| 3 | | 2.34 | | 2.0 | 8:17:00 AM |
| 4 | | 2.51 | | 2.0 | 8:19:00 AM |
| 5 | | 1.75 | | 1.0 | 8:21:00 AM |
| 6 | | 2.42 | | 2.0 | 8:24:00 AM |
| 9 | 2 | 2.80 | Meloxicam | 2.0 | 8:37:00 AM |
| 10 | | 2.51 | 0.1% | 2.0 | 8:39:00 AM |
| 11 | | 1.93 | | 1.0 | 8:41:00 AM |
| 12 | | 1.74 | | 1.0 | 8:43:00 AM |
| 13 | | 2.47 | | 2.0 | 8:33:00 AM |
| 14 | | 2.70 | | 2.0 | 8:35:00 AM |
| 7 | 3 | 2.55 | Tetracaine | 2.0 | 8:58:00 AM |
| 8 | | 2.17 | 5% + | 2.0 | 9:00:00 AM |
| 15 | | 2.31 | Meloxicam | 2.0 | 9:01:00 AM |
| 16 | | 2.38 | 0.1% | 2.0 | 9:04:00 AM |
| 17 | | 1.68 | | 1.0 | 9:07:00 AM |
| 18 | | 2.28 | | 2.0 | 9:09:00 AM |

TABLE 5
Schedule of Events

| Study | | |
|---|---|---|
| Day | Date | Event |
| −1 | 5 Dec. 2018 | Selected litters and confirmed suitability of selected animals. |
| | | Identified (uniquely numbered ear tag) individual piglets. Allocated |
| | | piglets to treatment groups. |
| 0 | 6 Dec. 2018 | Weighed piglets immediately prior to treatment. With piglets |
| | | restrained in a piglet cradle (Kerble, Shoof International), castrated |
| | | male piglets and applied treatment as per Table 4. |
| | | Collected blood samples via anterior vena-caval puncture at 15 (±5) |
| | | minutes from at least 4 animals in each group. |
| | | Collected blood samples via anterior vena-caval puncture at 30 |
| | | (±10) minutes from at least 4 animals in each group. |
| | | Collected blood samples via anterior vena-caval puncture at 1 hour |
| | | (±15 minutes) from at least 4 animals in each group. |
| | | Collected blood samples via anterior vena-caval puncture at 2 |
| | | (±0.5) hours from at least 4 animals in each group. |
| | | Collected blood samples via anterior vena-caval puncture at 4 |
| | | (±0.5) hours from at least 4 animals in each group. |
| | | Processed blood samples to extract plasma and stored individual |
| | | plasma samples in duplicate, frozen (~−18° C.). |
| Post- | 11 Dec. 2018 | Forwarded replicate 1 plasma samples to analytical laboratory. |
| Study | | |

TABLE 6
Study Animals

| | | | |
|---|---|---|---|
| Species: | Porcine | Number: | 18 |
| Breed: | Commercial | Source: | Commercial farm |
| | hybrid | | |
| Sex: | Male | | |
| Age: | 3-7 days at | Health & | No treatment with any |
| | treatment | special | product containing either |
| Method | Ear-tag | requirements: | tetracaine or meloxicam |
| of ID: | | | in lifetime. |

Health Management: The study animals were observed at each blood collection timepoint post-treatment. All piglets were normal except animal ID 16 which demonstrated an adverse reaction between ˜5-30 minutes post-treatment (see Table 7).

Adverse Events: Adverse events were recorded. One adverse event was encountered in this study, outlined in Table 7 below.

TABLE 7
Adverse Events

| Animal | | | |
|---|---|---|---|
| ID | Treatment | Event | Outcome |
| 16 | Tetracaine | At ~5 minutes post-treatment the piglet began to | Recovered |
| | 5% + | vocalise and laid in sternal recumbency with legs | |
| | Meloxicam | paddling. These signs continued until ~30 minutes | |
| | 0.1% | post-treatment. At the commencement of clinical | |
| | | signs, the piglet was assessed by a Veterinarian | |
| | | and returned to its litter mates and dam for | |
| | | constant monitoring until recovery. Apparent | |
| | | recovery occurred ~30 minutes post-treatment. | |
| | | This event was described as an adverse reaction to | |
| | | treatment. | |

Concurrent Medication: No concurrent medications were administered during the study.

Mortality: No mortality occurred during the study.

### Assessment of Effects

Blood Analysis: Blood samples were collected from allocated animals via anterior vena-caval puncture using 21G, 1.5″ needle attached to 10 mL syringes and transferred immediately into 5 mL lithium heparin blood tubes. Blood samples were processed for extraction of plasma on the day of collection, which for each sample was stored in duplicate, frozen (˜−18° C.). Replicate 1 plasma samples were forwarded frozen to a laboratory for tetracaine and meloxicam analyses after the conclusion of the animal phase.

### Statistical Analysis

Statistical analysis of derived plasma data was not performed in this study.

### Data Records

Study animals were dosed according to the application rates detailed in Table 4. No preparation of any of the IVPs was required prior to dosing. IVPs were administered using a 1 mL TRI-SOLFEN™ applicator with ball-point injection tip fitted to allow IVP deposition into the scrotal sac.

Castration was performed using a sterile scalpel as per the following process:
1. Piglet to be gently restrained (for a minimum of 30 seconds prior to incision) in Kerble piglet cradle in order to expose the ano-genital region of the piglet.
2. Only when piglet is settled, incise scrotum (including tunica) with one single transverse incision to expose and exteriorise each testis;
3. Immediately apply treatment (40% total dose to each side) to coat the exposed spermatic cord and the cut skin edge. Do not apply to run-off;
4. Wait 30 seconds;
5. The testis may then be removed by severing the cord as per routine procedure. Remove each testis as per normal procedure.
6. Apply the remaining 20% of the total dose evenly of the surface of the surgical wound.

### Results

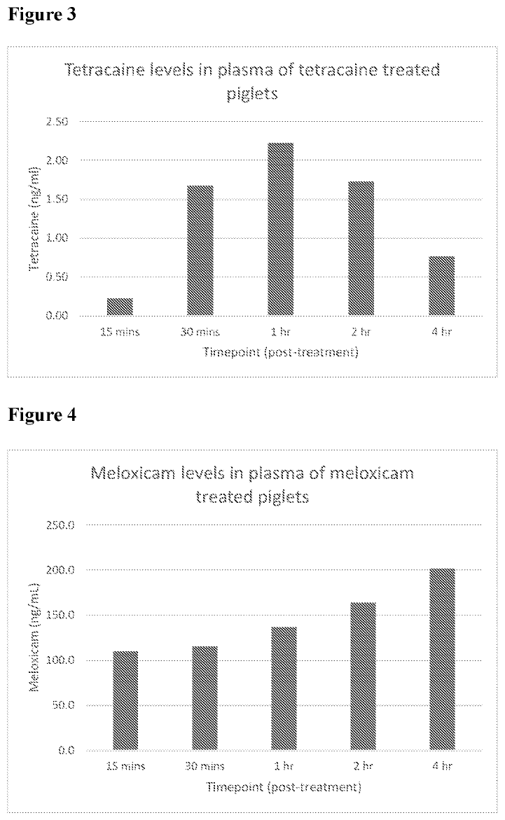

Individual animal raw data for tetracaine and meloxicam concentrations in plasma by timepoint relative to treatment is provided in Table 8. The data are summarised in FIGS. 3-7.

### Laboratory Results

TABLE 8
Tetracaine and meloxicam concentrations in porcine plasma.

| Animal | | | | | Tetracaine | Meloxicam |
|---|---|---|---|---|---|---|
| ID | Group | Treatment | Timepoint | | ng/mL | ng/mL |
| 1 | 1 | Tetracaine | 15 | min | 0.75 | — |
| 1 | 1 | Tetracaine | 30 | min | 1.96 | — |
| 1 | 1 | Tetracaine | 1 | hr | 0.00 | — |
| 1 | 1 | Tetracaine | 2 | hr | 0.14 | — |
| 1 | 1 | Tetracaine | 4 | hr | 0.00 | — |
| 2 | 1 | Tetracaine | 1 | hr | 0.00 | — |
| 2 | 1 | Tetracaine | 2 | hr | 0.99 | — |
| 3 | 1 | Tetracaine | 30 | min | 0.00 | — |
| 3 | 1 | Tetracaine | 1 | hr | 5.65 | — |
| 3 | 1 | Tetracaine | 2 | hr | 6.39 | — |
| 3 | 1 | Tetracaine | 4 | hr | 1.11 | — |
| 4 | 1 | Tetracaine | 15 | min | 0.00 | — |
| 4 | 1 | Tetracaine | 30 | min | 2.89 | — |
| 4 | 1 | Tetracaine | 1 | hr | 7.52 | — |
| 4 | 1 | Tetracaine | 2 | hr | 0.86 | — |
| 4 | 1 | Tetracaine | 4 | hr | 0.96 | — |
| 5 | 1 | Tetracaine | 15 | min | 0.15 | — |
| 5 | 1 | Tetracaine | 30 | min | 3.41 | — |
| 5 | 1 | Tetracaine | 1 | hr | 0.19 | — |
| 5 | 1 | Tetracaine | 2 | hr | 1.86 | — |
| 5 | 1 | Tetracaine | 4 | hr | 1.44 | — |
| 6 | 1 | Tetracaine | 15 | min | 0.00 | — |
| 6 | 1 | Tetracaine | 30 | min | 0.11 | — |
| 6 | 1 | Tetracaine | 1 | hr | 0.00 | — |
| 6 | 1 | Tetracaine | 2 | hr | 0.14 | — |
| 6 | 1 | Tetracaine | 4 | hr | 0.30 | — |
| 9 | 2 | Meloxicam | 15 | min | — | 86.9 |
| 9 | 2 | Meloxicam | 30 | min | — | 109.4 |
| 9 | 2 | Meloxicam | 2 | hr | — | 125.7 |
| 9 | 2 | Meloxicam | 4 | hr | — | 100.9 |
| 10 | 2 | Meloxicam | 15 | min | — | 144.8 |
| 10 | 2 | Meloxicam | 30 | min | — | 166.0 |
| 10 | 2 | Meloxicam | 1 | hr | — | 189.6 |
| 10 | 2 | Meloxicam | 4 | hr | — | 458.7 |
| 11 | 2 | Meloxicam | 30 | min | — | 94.6 |
| 11 | 2 | Meloxicam | 1 | hr | — | 102.9 |
| 11 | 2 | Meloxicam | 2 | hr | — | 126.7 |
| 11 | 2 | Meloxicam | 4 | hr | — | 63.3 |
| 12 | 2 | Meloxicam | 15 | min | — | 119.8 |
| 12 | 2 | Meloxicam | 30 | min | — | 101.3 |
| 12 | 2 | Meloxicam | 4 | hr | — | 230.0 |
| 13 | 2 | Meloxicam | 15 | min | — | 81.8 |
| 13 | 2 | Meloxicam | 2 | hr | — | 238.9 |
| 13 | 2 | Meloxicam | 4 | hr | — | 244.6 |
| 14 | 2 | Meloxicam | 15 | min | — | 88.2 |
| 14 | 2 | Meloxicam | 30 | min | — | 104.8 |
| 14 | 2 | Meloxicam | 1 | hr | — | 118.0 |
| 14 | 2 | Meloxicam | 4 | hr | — | 113.2 |
| 7 | 3 | Tetracaine + | 30 | min | 0.56 | 48.9 |
| | | Meloxicam | | | | |
| 7 | 3 | Tetracaine + | 1 | hr | 3.52 | 49.1 |
| | | Meloxicam | | | | |
| 7 | 3 | Tetracaine + | 2 | hr | 0.00 | 75.3 |
| | | Meloxicam | | | | |
| 8 | 3 | Tetracaine + | 15 | min | 0.00 | 44.4 |
| | | Meloxicam | | | | |
| 8 | 3 | Tetracaine + | 1 | hr | 2.45 | 52.0 |
| | | Meloxicam | | | | |
| 8 | 3 | Tetracaine + | 2 | hr | 1.02 | 63.4 |
| | | Meloxicam | | | | |
| 8 | 3 | Tetracaine + | 4 | hr | 0.00 | 66.0 |
| | | Meloxicam | | | | |
| 15 | 3 | Tetracaine + | 15 | min | 1.90 | 40.6 |
| | | Meloxicam | | | | |
| 15 | 3 | Tetracaine + | 30 | min | 1.35 | 45.2 |
| | | Meloxicam | | | | |
| 15 | 3 | Tetracaine + | 1 | hr | 0.90 | 41.7 |
| | | Meloxicam | | | | |
| 15 | 3 | Tetracaine + | 2 | hr | 0.30 | 42.3 |
| | | Meloxicam | | | | |
| 15 | 3 | Tetracaine + | 4 | hr | 0.00 | 34.6 |
| | | Meloxicam | | | | |
| 17 | 3 | Tetracaine + | 15 | min | 0.16 | 42.6 |
| | | Meloxicam | | | | |
| 17 | 3 | Tetracaine + | 30 | min | 1.52 | 48.8 |
| | | Meloxicam | | | | |
| 17 | 3 | Tetracaine + | 2 | hr | ns | ns |
| | | Meloxicam | | | | |
| 17 | 3 | Tetracaine + | 4 | hr | ns | ns |
| | | Meloxicam | | | | |
| 18 | 3 | Tetracaine + | 15 | min | 0.00 | 38.0 |
| | | Meloxicam | | | | |
| 18 | 3 | Tetracaine + | 30 | min | 0.00 | 45.5 |
| | | Meloxicam | | | | |
| 18 | 3 | Tetracaine + | 1 | hr | 0.00 | 54.9 |
| | | Meloxicam | | | | |
| 18 | 3 | Tetracaine + | 2 | hr | 0.00 | 262.9 |
| | | Meloxicam | | | | |
| 18 | 3 | Tetracaine + | 4 | hr | 0.00 | 80.1 |

Meloxicam

ns = no sample (blood collected from wrong piglet)

### Assessment of Effects

Blood Analysis: Blood Samples were Collected from Allocated Animals Via Anterior

Very low plasma tetracaine levels were confirmed in tetracaine treated piglets, (consistent with rapid metabolism via plasma esterases). For piglets treated with 5% tetracaine (Group 1), plasma levels peaked at 1 hour post-treatment at 2 ng/ml (FIG. 3). For piglets treated with 5% tetracaine in the combination product (5% tetracaine+0.1% meloxicam) (Group 3), plasma tetracaine levels similarly peaked at 1 hour post-treatment (FIG. 3); however, at an even lower level than in the animals treated with the single active 5% tetracaine product (Group 1) and was not quantifiable by 4 hours post-treatment. A piglet in this group had an adverse event. (This is considered to be possibly due to a hypersensitivity reaction to PABA, the predominant metabolite of tetracaine).

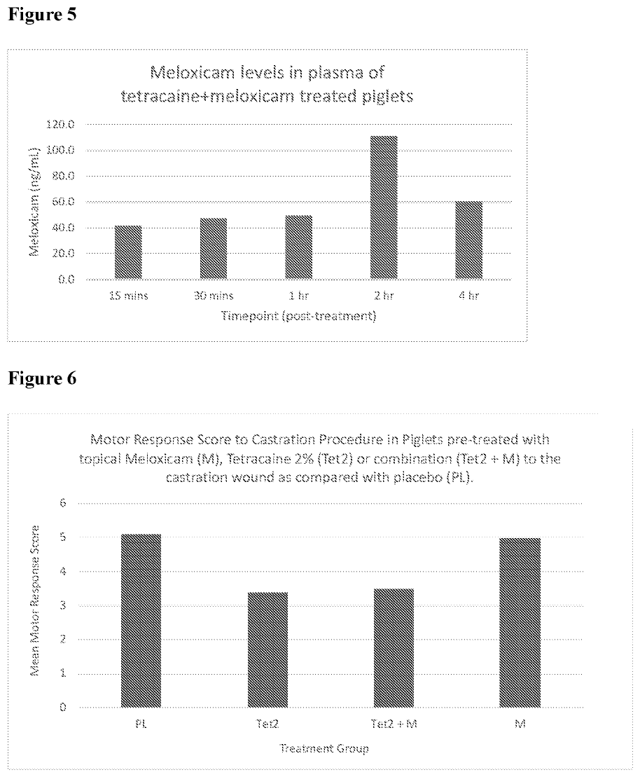

Higher and sustained plasma levels of meloxicam (100-200 ng/ml) were seen in piglets treated with 0.1% meloxicam (Group 2). The peak plasma level was recorded at the 4 hour time point (FIG. 4). For piglets treated with 0.1% meloxicam in the combination product (5% tetracaine+0.1% meloxicam) (Group 3), plasma levels of meloxicam peaked at 2 hours post-treatment (FIG. 5) and at a lower level than the 4 hour post-treatment peak achieved by piglets treated with the single active 0.1% meloxicam formulation (Group 2). Hence, the maximum plasma concentrations of tetracaine and/or meloxicam were highest in piglets treated with single active formulations in comparison to the combination formulation.

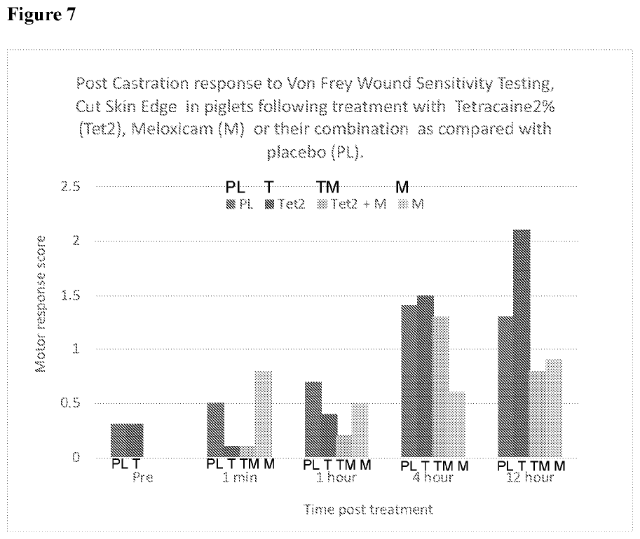

Study 2: A follow-up study confirmed efficacy of meloxicam analgesic effects when applied to piglets via topical wound application either alone or in combination with tetracaine 2%. Piglets were treated with placebo, meloxicam 0.1%, tetracaine 2% or a combination of tetracaine 2% and meloxicam 1%, otherwise formulated as above, and examined for clinical efficacy via documentation of motor response scores to a) the procedure of castration, and b) pre- and post-operative mechanical sensory testing of the wound site using Von-Frey filament testing. Results indicated a significant reduction in motor response scores during the procedure and at 1 minute following the procedure in tetracaine treated piglets (consistent with rapid onset local anaesthesia), which was not evident in meloxicam-only treated piglets (FIGS. 6 and 7). In tetracaine treated piglets, anaesthetic effects were still evident at 1 hour, however wore off there-after, with evidence of rebound hyperaesthesia developing by 12 hours. Response scores in meloxicam treated piglets did not differ significantly from placebo treated piglets during the procedure or at 1 minute following the procedure, however were below placebo treated piglets at 1, 4 and 12 hours indicative of analgesia secondary to anti-inflammatory effects during the post-operative period (FIG. 7). Analgesic effects of meloxicam delivered via this technique thus appeared to be more prolonged than following IM delivery as demonstrated in the trials of Keita et al., et al (although measured using different techniques; Keita, Alassane, Eric Pagot, Armelle Prunier, and Christian Guidarini. “Pre-Emptive Meloxicam for Postoperative Analgesia in Piglets Undergoing Surgical Castration.” Veterinary Anaesthesia and Analgesia 37, no. 4 (July 2010): 367-74. https://doi.org/10.1111/j.1467-2995.2010.00546.x.). Furthermore, a combination of early anaesthetic effects (due to tetracaine) and later analgesic effects (due to meloxicam) were seen in the piglets treated with combination therapy, in whom motor response scores were below those of placebo treated piglets during castration, and at 1 minute 1 hour and 12 hours following the procedure (FIGS. 6 and 7).

### CONCLUDING REMARKS

In some embodiments, an innovative and novel method of systemic drug delivery and combination local and systemic drug delivery via topical wound application is presented along with compositions for topical application to wounds that deliver systemic, or combination local and systemic pain relieving effects.

Advantages of some embodiments of the present invention as exemplified include providing a new “all-in-one” method of mitigating wound pain simultaneously addressing local neural and systemic inflammatory pain generating mechanisms, that can be used to reduce or minimise pain in a large variety of wound types or wound situations (front-line military, on-farm etc) in which other means of providing such therapy or combination therapy (such as via injections or multimodal strategies) are not currently considered or used by virtue of being too impractical, dangerous, complex or costly.

Other advantages of some embodiments of the pain relieving composition of the present invention include the fact that it can be directly applied to wounds obviating the need for other methods of systemic administration such as oral, IM or IV therapy. This provides a novel alternative for drug delivery in situations (such as shock) where oral, IM or IV therapy may be ineffective or difficult to achieve, and/or it may reduce pain and stress involved with needles or double handling such as for pain management of wounds in infants, children and animal husbandry procedures.

Throughout this specification, unless in the context of usage an alternative interpretation is required, the term “comprise” (and variants thereof such as “comprising” and “comprised”) denotes the inclusion of a stated integer or integers but does not exclude the presence of another integer or other integers.

Any reference to publications cited in this specification is not an admission that the disclosures constitute common general knowledge in Australia or in other countries.

It will be appreciated by one of skill in the art that many changes can be made to the composition and uses exemplified above without departing from the broad ambit and scope of the invention.

1. A topical drug delivery formulation, formulated to carry at least one drug, be applied to or administered via an open wound of a subject, and enable systemic absorption of the at least one drug through the open wound to provide a therapeutic effect for a predetermined period of time

2. A topical drug delivery composition comprising:
at least one drug; and
a drug delivery formulation, formulated to carry the at least one drug, be applied to or administered via an open wound of a subject, and enable systemic absorption of the at least one drug through the open wound to provide a therapeutic effect for a predetermined period of time.

3. A method of delivering at least one drug systemically to a subject via an open wound of said subject, said method comprising the step of topically applying to or administering via the open wound the drug delivery formulation of claim 1 to provide a therapeutic effect for a predetermined period of time.

4-18. (canceled)

19. The drug delivery formulation according to claim 1, wherein the at least one drug is one or more drugs selected from the group consisting of: an analgesic, anaesthetic, sedative, narcotic, anxiolytic antibiotic, anti-microbial, antifungal or anti-parasitic agent, antibody, coagulant, anticoagulant, haemostatic agent, vaccine, immune globulin, vasopressor, inotrope, alpha blocker, beta blocker, antiarrhythmic, antihistamine, antiproliferative, cytokine, cytotoxin, growth factor, interferon, steroid, hormone, lipid, demineralized bone or bone morphogenetic protein, cartilage inducing factor, oligonucleotide, polymer, polysaccharide, polypeptide, protease inhibitor, vitamin, mineral, and antiseptic agent.

20. The drug delivery composition of claim 2, wherein the at least one drug is one or more drugs selected from the group consisting of: an analgesic, anaesthetic, sedative, narcotic, anxiolytic antibiotic, anti-microbial, antifungal or anti-parasitic agent, antibody, coagulant, anticoagulant, haemostatic agent, vaccine, immune globulin, vasopressor, inotrope, alpha blocker, beta blocker, antiarrhythmic, antihistamine, antiproliferative, cytokine, cytotoxin, growth factor, interferon, steroid, hormone, lipid, demineralized bone or bone morphogenetic protein, cartilage inducing factor, oligonucleotide, polymer, polysaccharide, polypeptide, protease inhibitor, vitamin, mineral, and antiseptic agent.

21. The drug delivery formulation of claim 19, wherein the at least one drug comprises an anaesthetic agent and/or an analgesic agent.

22. The drug delivery composition of claim 20, wherein the at least one drug comprises an anaesthetic agent and/or an analgesic agent.

23. The drug delivery formulation of claim 21, wherein the at least one drug comprises tetracaine and meloxicam.

24. The drug delivery composition of claim 22, wherein the at least one drug comprises tetracaine and meloxicam.

25. The drug delivery formulation of claim 23, wherein the at least one drug comprises about 0.5-10 weight/volume % tetracaine and about 0.1-1 weight/volume % meloxicam.

26. The drug delivery composition of claim 24, wherein the at least one drug comprises about 0.5-10 weight/volume % tetracaine and about 0.1-1 weight/volume % meloxicam.

27. The drug delivery formulation of claim 1, wherein the drug delivery formulation forms an adhesive gel when applied to the wound.

28. The drug delivery composition of claim 2, wherein the drug delivery composition or drug delivery formulation forms an adhesive gel when applied to the wound.

29. The drug delivery formulation of claim 1, wherein the drug delivery formulation is in the form of a liquid that thickens to an adhesive gel when reacting with physiological fluids of the open wound.

30. The drug delivery composition of claim 2, wherein the drug delivery composition or drug delivery formulation is in the form of a liquid that thickens to an adhesive gel when reacting with physiological fluids of the open wound.

31. The drug delivery formulation of claim 1, wherein the drug delivery formulation is biocompatible and absorbable such that the formulation does not require removal from the wound.

32. The drug delivery composition of claim 2, wherein the drug delivery composition or drug delivery formulation is biocompatible and absorbable such that they do not require removal from the wound.

33. A method of delivering at least one drug systemically to a subject via an open wound of said subject, said method comprising the step of topically applying to or administering via the open wound the drug delivery composition of claim 2 to provide a therapeutic effect for a predetermined period of time.

34. A method for administering pain relief to a large number of animals for a husbandry procedure in a short period of time, said method comprising the steps of:
creating an open wound on each said animal in accordance with the husbandry procedure; and
applying to the wound of each said animal the drug delivery formulation of claim 1 to provide a therapeutic effect for a predetermined period of time.

35. A method for administering pain relief to a large number of animals for a husbandry procedure in a short period of time, said method comprising the steps of:
creating an open wound on each said animal in accordance with the husbandry procedure; and
applying to the wound of each said animal the drug delivery composition of claim 2 to provide a therapeutic effect for a predetermined period of time.
